(19) United States
(12) Patent Application Publication
(10) Pub. No.: US 2021/0076947 A1
(43) Pub. Date: Mar. 18, 2021

(54) DEVICE FOR REMOTELY MONITORING A PATIENT WITH CHRONIC HEART FAILURE, AND ASSOCIATED SYSTEM AND METHOD

(71) Applicant: EDEVICE, MERIGNAC (FR)

(72) Inventors: Pierre RAPHAEL, TOURS (FR); Patrick JOURDAIN, PONTOISE (FR)

(21) Appl. No.: 16/961,316

(22) PCT Filed: Jan. 28, 2019
(86) PCT No.: PCT/FR2019/050173

§ 371 Date: Jul. 10, 2020

(30) Foreign Application Priority Data
Jan. 30, 2018 (FR) 1850760

(51) Int. Cl. A61B 5/0205; A61B 5/1455; A61B 5/00; G01G 19/44; G16H 40/67; G16H 10/60; G16H 50/30; G16H 50/70

(52) U.S. Cl. CPC A61B 5/0205; A61B 5/14551; A61B 5/002; A61B 5/746; A61B 5/0022; A61B 5/024; G16H 40/67; G16H 10/60; G16H 50/30; G16H 50/70; G01G 19/44

(57) ABSTRACT

The invention relates to a device (3) for remotely monitoring a patient with chronic heart failure, said device comprising: a first communication interface (11) suitable for communicating with a weighing scales (5) in order to obtain a measurement of the patient's weight; a second communication interface (13) suitable for communicating with an oxygen meter (7) in order to obtain measurements of the patient's heart rate and blood oxygen saturation level; a third communication interface (15) suitable for transmitting a warning to the patient; and a computer (19) connected to the three interfaces (11, 13, 14), comprising a memory (21) suitable for storing the daily measurements collected, said computer being configured to generate a warning when the collected measurements indicate the likely onset of cardiac decompensation.

### TECHNICAL FIELD

The present invention relates to a device for remotely monitoring a patient suffering from chronic heart failure, and to a remote monitoring system comprising such a device and a remote monitoring method using such a device.

### STATE OF THE ART

Chronic heart failure is a sickness which has a number of specific features. It is both common and serious. Chronic heart failure affects approximately 1% of the population in the developed countries. That includes almost 800 000 patients in France. It is a sickness which progresses in particular because of the inexorable aging of the population with a number of hospitalizations which regularly increases (14% increase in France between 2002 and 2008). This trend is confirmed in other countries like the USA. It is a sickness which is also associated with high mortality and also with a high repeat hospitalization rate. That is partly due to the “per se” severity of the sickness but also to a non-optimal management of the sickness and to an ill-informed education of the patients and of the carers. Thus, most of the hospitalized patients have shown clinical signs of decompensation and have done so up to 5 days before hospitalization. A good management of these signs could have prevented the patients from going to the emergency rooms and emergency hospitalization, through outpatient management and care.

It is from this observation that the benefit of using telemedicine for chronic heart failure emerges. Telemedicine is the monitoring of clinical or biological data in the home of the patient with the transfer of these data remotely either to a dedicated structure often managed by nurses, or to the treating generalist doctor or the cardiologist.

Many types of data have been studied and tracked in the scientific and medical literature for tracking the state of health. Mention may be made of data relating to the state of health of the patient, clinical data such as, for example, the weight and its variations, the arterial pressure, the heart rate, the physical activity, etc., or biological data such as by impedance measurement, by direct measurement of intracardial pressure, or by electrocardiography, etc. Unfortunately, the results are extremely variable according to the studies and because of this, to date, the use of telemedicine is not validated and recommended in the management of chronic heart failure.

Furthermore, the use of telemedicine has shown many limitations underscored by many studies. In particular, the low sensitivity and the specificity of the clinical signs often generate alerts which makes management thereof by the patient and the doctor difficult. Telemedicine is also costly with complex methodology. Furthermore, it raises medico-legal problems relating to the liability of those interpreting the remote telemedicine data.

There is therefore a real need for a device for remotely monitoring patients afflicted with chronic heart failure that mitigates these defects, drawbacks and obstacles of the prior art, in particular for a device that is relatively inexpensive and sufficiently reliable in its alerts to minimize false alarms.

### SUMMARY OF THE INVENTION

To resolve one or more of the abovementioned drawbacks, the invention relates, according to a first aspect, to a device for remotely monitoring a patient suffering from chronic heart failure, said device comprises:
a first communication interface adapted to communicate with a weighing scale in order to obtain a measurement of the weight of the patient;
a second communication interface adapted to communicate with an oxygen meter in order to obtain measurements of the heart rate and of the blood oxygen saturation level of the patient;
a third communication interface adapted to transmit an alert to the patient; and
a computer connected to the three interfaces, comprising a storage memory adapted to store the measurements obtained, the computer being adapted to trigger an alert when the measurements obtained indicate a probable onset of cardiac decompensation in the patient;
   the computer being configured to:
obtain a nominal weight of the patient and predetermined values comprising:
   a first weight limit increase;
   a second weight limit increase strictly less than the first weight limit increase;
   a first blood oxygen saturation level;
   a second blood oxygen saturation level strictly greater than the first blood oxygen saturation level;
   a first heart rate; and
   a second heart rate strictly less than the first heart rate,
determine, from the measurements obtained, whether any one of the following first conditions is fulfilled:
   the weight has increased more than the first weight limit increase relative to the nominal weight of the patient;
   the weight has increased, between two measurements taken over a predetermined period strictly greater than 2 days, by a value strictly greater than the first weight limit increase;
   the blood oxygen saturation level is strictly less than the first blood oxygen saturation level;
   the heart rate is strictly greater than the first heart rate;
determine that an onset of cardiac decompensation is probable when at least one of the first conditions is fulfilled;
determine, from the measurements obtained, whether any one of the following second conditions is fulfilled:
   the weight has increased between two measurements taken over 2 consecutive days, by a value strictly greater than the second weight limit increase;
   the blood oxygen saturation level is strictly less than the second blood oxygen saturation level;
   the heart rate is strictly greater than the second heart rate,
determine that an onset of cardiac decompensation is probable when two of the second conditions are fulfilled simultaneously, while none of the first conditions is fulfilled.

Such a device has the advantage of using measurement means, weighing scale and oxygen meter, that are relatively inexpensive, and of increasing the reliability of the triggering of the alert by the combination of three different types of measurement. The algorithm is simple and generic. Furthermore, the use of different thresholds for these three types of measurement depending on whether to proceed with the verification of the first conditions or of the second conditions makes it possible to increase the reliability of the triggering in that unwanted triggerings can be avoided while guaranteeing that the alerts that are triggered are relevant.

Features or particular embodiments, that can be used alone or in combination, according to which:
the first weight limit increase is equal to 4 kg; and
the second weight limit increase is equal to 2 kg;
the predetermined period is 4 days; and
the first blood oxygen saturation level is equal to 90%; and
the second blood oxygen saturation level is equal to 95%; and
the first heart rate is equal to 110 beats/min; and
the second heart rate is equal to 90 beats/min;
the remote monitoring device comprises a clock associated with a human-machine interface for reminding the patient to take his or her daily measurements within a predetermined time band; and/or
the remote monitoring device comprises a fourth communication interface adapted to transmitting the measurements to a remote server.

In a second aspect of the invention, a system for remotely monitoring a patient suffering from heart failure comprises:
a remote monitoring device as described in this document;
a weighing scale;
an oxygen meter; the weighing scale and the oxygen meter being connected to the remote monitoring device; and
a remote server connected to the remote monitoring device and adapted to obtain, store and display the history of the measurements of the patient.

In a third aspect of the invention, a method for remotely monitoring a patient suffering from heart failure, the method being intended to be implemented by a remote monitoring device and comprising the following steps:
obtaining, via a first communication interface with a weighing scale, measurements of the weight of the patient;
obtaining, via a second communication interface with an oxygen meter, measurements of the heart rate and of the blood oxygen saturation level of the patient;
triggering of an alert when the measurements obtained indicate a probable onset of cardiac decompensation in the patient;
obtaining predetermined values comprising:
   a first weight limit increase;
   a second weight limit increase strictly less than the first weight limit increase;
   a first blood oxygen saturation level;
   a second blood oxygen saturation level strictly greater than the first blood oxygen saturation level;
   a first heart rate; and
   a second heart rate strictly less than the first heart rate,
determining, from the obtained measurements, whether any one of the following first conditions is fulfilled:
   the weight has increased by a value strictly greater than the first weight limit increase relative to the nominal weight of the patient;
   the weight has increased, between two measurements taken over a predetermined period strictly greater than 2 days, by a value strictly greater than the first weight limit increase;
   the blood oxygen saturation level is strictly less than the first blood oxygen saturation level;
   the heart rate is strictly greater than the first heart rate;
triggering of an alert signaling that an onset of cardiac decompensation is probable when at least one of the first conditions is fulfilled;
determining, from the obtained measurements, whether any one of the following second conditions is fulfilled:
   the weight has increased between two measurements taken over 2 consecutive days, by a value strictly greater than the second weight limit increase;
   the blood oxygen saturation level is strictly less than the second blood oxygen saturation level;
   the heart rate is strictly greater than the second heart rate;
triggering of an alert signaling that an onset of cardiac decompensation is probable when two of the second conditions are fulfilled simultaneously, while none of the first conditions is fulfilled.

More generally, the method for remotely monitoring a patient suffering from heart failure comprises the following steps:
measurement, each day in similar conditions, of the weight, of the heart rate and of the blood oxygen saturation level of the patient;
triggering of an alert when the measurements obtained indicate a probable onset of cardiac decompensation in the patient.

Features or particular embodiments of the method, that can be used alone or in combination, are as follows:
the first weight limit increase is equal to 4 kg; and
the second weight limit increase is equal to 2 kg;
the predetermined period is 4 days; and
the first blood oxygen saturation level is equal to 90%; and
the second blood oxygen saturation level is equal to 95%; and
the first heart rate is equal to 110 beats/min; and
the second heart rate is equal to 90 beats/min.

Another aspect of the invention relates to a computer program to be installed in a remote monitoring device, comprising instructions for implementing the steps of a method as defined above, notably when the program is run by a computation unit or a data processor or computer of a monitoring unit or device as described in this document.

### BRIEF DESCRIPTION OF THE FIGURES

The invention will be better understood on reading the following description, given purely as an example, and by referring to the attached figures in which:

FIG. 1 represents a remote monitoring system according to an embodiment of the invention;

FIG. 2 represents an architecture of a remote monitoring device, an element of the system of FIG. 1; and

FIG. 3 represents a flow diagram of a method implemented by the system of FIG. 1.

### EMBODIMENTS

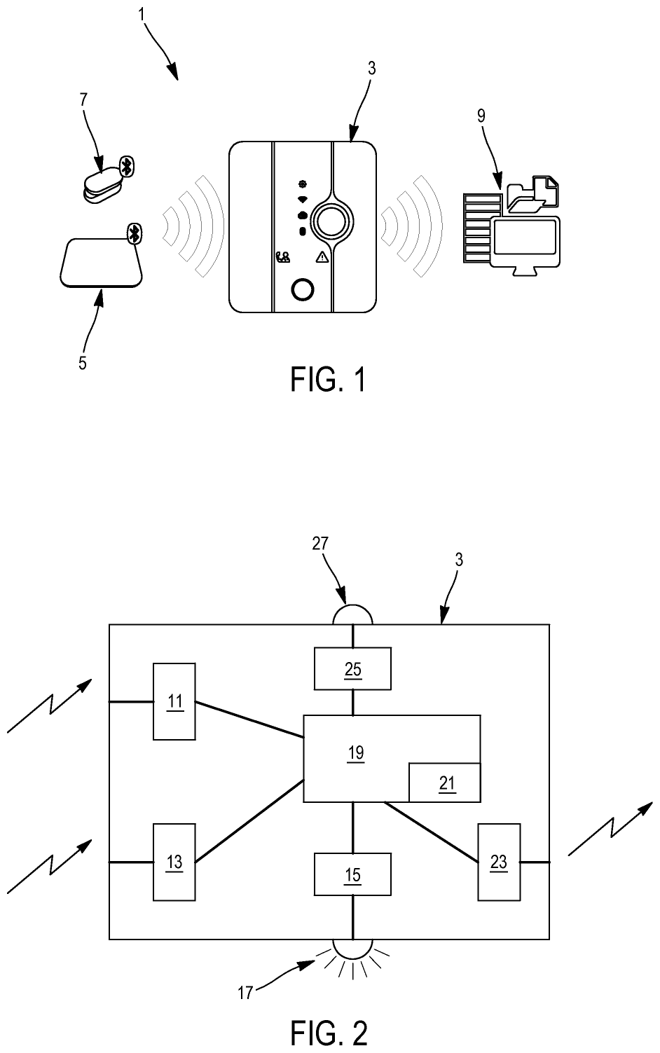

Referring to FIG. 1, a system 1 for remotely monitoring a patient suffering from chronic heart failure comprises a remote monitoring device 3 connected on one side to a weighing scale 5 and an oxygen meter 7 and on the other side to a remotely installed care management server 9.

The remote monitoring device 3, the weighing scale 5 and the oxygen meter 7 are installed in the home of the patient while the care management server 9 is installed with a service provider ensuring the management of the system.

The connection of the remote monitoring device 3 with the weighing scale 5 and the oxygen meter 7 is set up preferably by a short-range wireless link of Bluetooth type.

The connection of the remote monitoring device 3 with the care management server 9 is ensured by a WiFi link with a modem-router (not represented) which communicates with the internet network.

In more detail, the remote monitoring device 3 comprises, FIG. 2, a first communication interface 11 adapted to communicate with the weighing scale 5 in order to obtain a measurement of the weight of the patient.

A second communication interface 13 is adapted to communicate with the oxygen meter in order to obtain measurements of the heart rate and of the blood oxygen saturation level of the patient.

The remote monitoring device 3 also comprises a third communication interface 15 adapted to transmit an alert to the patient. For example, as illustrated in FIG. 2, the alert is transmitted by a light indicator 17 which is off in normal mode and which flashes in the event of an alert.

A computer 19 is connected to the three interfaces 11, 13, 15. It comprises a storage memory 21 adapted to store the measurements obtained, the computer being adapted to generate an alert when the measurements obtained indicate a probable onset of cardiac decompensation in the patient.

The computer 19 is also connected to a fourth communication interface 23 adapted to transmit the measurements obtained to the server 9.

The remote monitoring device 3 also comprises a clock 25 associated with a human-machine interface 27 for reminding the patient to take his or her measurements within a predetermined time band.

As for the alert, this human-machine interface 27 is for example a light display.

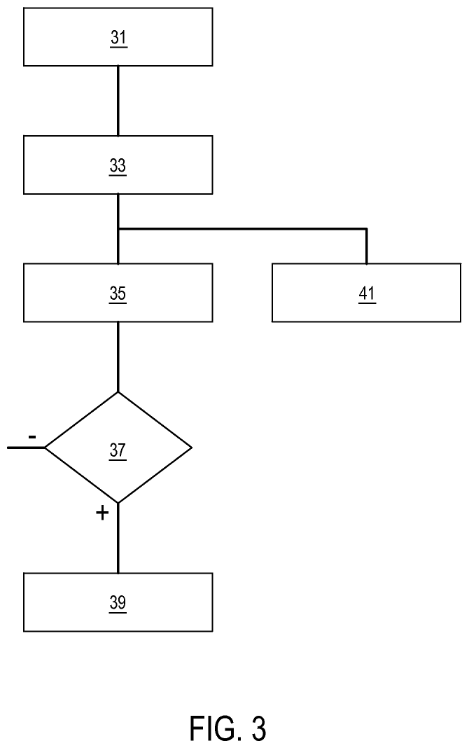

The operation of the remote monitoring system 1 and, more particularly, that of the remote monitoring device 3, will now be described in relation to FIG. 3.

First of all, alert thresholds on the various measurements, of weight, heart rate and blood oxygen saturation level, are predefined, in a step 31.

Concerning the weight, a first weight limit increase and a second weight limit increase strictly less than the first weight limit increase are defined.

For example, it will be defined that the first weight limit increase is 4 kg and the second weight limit increase is 2 kg.

A rapid increase in the weight of the patient above these two limit values can be the sign of a water retention episode.

Likewise, a first blood oxygen saturation level and a second blood oxygen saturation level strictly greater than the first blood oxygen saturation level are defined.

For example, the first blood oxygen saturation level is equal to 90% and the second blood oxygen saturation level is equal to 95%.

A low blood oxygen saturation level can be the first sign of an acute edema of the lung.

Finally, a first heart rate and a second heart rate strictly less than the first heart rate are defined.

For example, the first heart rate is equal to 110 beats/min and the second heart rate is equal to 90 beats/min.

After installation of the various elements of the remote monitoring system 1 at the home of the patient and verification of the correct operation and that the various connections are made without difficulty, the monitoring protocol is put in place.

Every day, the patient measures, in a step 33, his or her weight, heart rate and blood oxygen saturation level.

For the measurements to be comparable, the measurement session must be conducted always in the same conditions, preferably daily. Thus, it is preferable for it to take place at sunrise, with fasting and after having gone to the toilet.

That therefore defines a time band for the taking of the measurements. To guarantee the effectiveness of the operation, the remote monitoring device 3 uses its clock 25 associated with the human-machine interface 27 to remind the patient to take the measurements.

Moreover, the first weight measurement, or the first measurement after a period without measurement of more than 4 days or a first measurement obtained after a resetting of the monitoring device and/or of its computer, can be used to define the nominal weight of the patient.

Once the measurements have been done and obtained by the remote monitoring device 3, the computer 19 analyzes them, in a step 35, according to the following decision rules.

It is probable that there is an onset of cardiac decompensation if one of the following first conditions is fulfilled:
the weight has increased by a value strictly greater than the first weight limit increase relative to the nominal weight of the patient;
the weight has increased, between two measurements taken over a predetermined period strictly greater than 2 days (this first period is for example equal to 4 days), by a value strictly greater than the first weight limit increase;
the blood oxygen saturation level is strictly less than the first blood oxygen saturation level;
the heart rate is strictly greater than the first heart rate; or,

it is probable that there is an onset of cardiac decompensation if two of the following second conditions are fulfilled simultaneously, while none of the preceding first conditions is fulfilled:
the weight has increased between two measurements, taken over a second period equal to 2 consecutive days, by a value strictly greater than the second weight limit increase;
the blood oxygen saturation level is strictly less than the second blood oxygen saturation level;
the heart rate is strictly greater than the second heart rate.

By using the above numeric data, and by considering that the predetermined period indicated above is 4 days, these rules are translated into:
the weight has increased by more than 4 kg relative to the nominal weight of the patient;
the weight has increased by more than 4 kg in 4 days;
the blood oxygen saturation level is strictly less than 90%;
the heart rate is strictly greater than 110 beats/min; or
two of the following conditions are fulfilled simultaneously:
the weight has increased by more than 2 kg in 2 days;
the blood oxygen saturation level is strictly less than 95%;
the heart rate is strictly greater than 90 beats/min.

If, according to the above rules, the computer 19 considers, in a step 37, that an onset of heart failure is probable, then it triggers, in a step 39, an alert for the patient.

In parallel with this analysis, the measurements are transmitted, in a step 41, to the server 9.

It is noted that the periods used for the verification of the first conditions and second conditions can be adapted. The first period (4 days in the preceding examples) used for the verification of the first conditions is greater than the second period (2 days in the preceding examples) used for the verification of the second conditions.

Receiving the alert, the patient can then go to his or her doctor, or call the doctor. Said doctor has access to the measurements stored on the server 9 and is then able to make a care decision: hospitalization, more comprehensive consultation, etc., based on the diagnosis that can be posited.

It is important to note that the system is not intended to replace the usual medical management of the patient but to alert the patient for the latter to have an active procedure for managing his or her state of health.

The invention has been illustrated and described in detail in the drawings and the above description. The latter should be considered illustrative and given as an example and not as limiting the invention to just this description. Many variant embodiments are possible.

For example, the connection between the remote monitoring device 3 and the weighing scale 5 or the oxygen meter 7 can be provided by a wireless link of WiFi type or else by a wired electrical link such as a serial link. Although less practical, it is also possible to envisage having the data entered manually by the patient.

Likewise, the connection between the remote monitoring device 3 and the care management server 9 can be a wired link of Ethernet type or else a wireless link via a telephone network according to a standard of 3G or 4G type. Depending on the operating environment, the person skilled in the art will be able to choose the most appropriate mode of connection for transferring the data reliably and inexpensively.

Likewise, the patient can be alerted by the remote monitoring device by different sound, touch or visual means. For example, the alert can be transmitted to a connected watch worn by the patient and which will generate a vibration in his or her wrist.

In the claims, the word “comprising” does not preclude other elements and the indefinite article “a/an” does not preclude a plurality.

1. A device for remotely monitoring a patient suffering from chronic heart failure, said device comprising:
a first communication interface configured to communicate with a weighing scale in order to obtain a measurement of the weight of the patient;
a second communication interface configured to communicate with an oxygen meter in order to obtain measurements of the heart rate and of the blood oxygen saturation level of the patient;
a third communication interface configured to transmit an alert to the patient; and
a computer connected to the three interfaces, comprising a storage memory configured to store the measurements obtained, the computer being configured to trigger an alert when the measurements obtained indicate a probable onset of cardiac decompensation in the patient; the computer being configured to:
obtain a nominal weight of the patient and predetermined values comprising:
a first weight limit increase;
a second weight limit increase strictly less than the first weight limit increase;
a first blood oxygen saturation level;
a second blood oxygen saturation level strictly greater than the first blood oxygen saturation level;
a first heart rate; and
a second heart rate strictly less than the first heart rate,
determine, from the measurements obtained, whether any one of the following first conditions is fulfilled:
the weight has increased more than the first weight limit increase relative to the nominal weight of the patient;
the weight has increased, between two measurements taken over a predetermined period strictly greater than 2 days, by a value strictly greater than the first weight limit increase;
the blood oxygen saturation level is strictly less than the first blood oxygen saturation level;
the heart rate is strictly greater than the first heart rate;
determine that an onset of cardiac decompensation is probable when at least one of the first conditions is fulfilled;
determine, from the measurements obtained, whether any one of the following second conditions is fulfilled:
the weight has increased between two measurements taken over 2 consecutive days by a value strictly greater than the second weight limit increase;
the blood oxygen saturation level is strictly less than the second blood oxygen saturation level;
the heart rate is strictly greater than the second heart rate,
determine that an onset of cardiac decompensation is probable when two of the second conditions are fulfilled simultaneously, while none of the first conditions is fulfilled.

2. The device as claimed in claim 1, wherein the nominal weight corresponds to a first weight measurement or a first measurement after a resetting of the device or a first measurement after a period without measurement of more than 4 days.

3. The device as claimed in claim 1, wherein
the first weight limit increase is equal to 4 kg;
the second weight limit increase is equal to 2 kg;
the predetermined period is 4 days;
the first blood oxygen saturation level is equal to 90%;
the second blood oxygen saturation level is equal to 95%;
the first heart rate is equal to 110 beats/min;
the second heart rate is equal to 90 beats/min.

4. The device as claimed in claim 1, wherein that it comprises a clock associated with a human-machine interface for reminding the patient to take his or her measurements each day within a predetermined time band.

5. The device as claimed in claim 1, wherein it comprises a fourth communication interface configured to transmit the measurements to a remote server.

6. A system for remotely monitoring a patient suffering from heart failure comprising:
a remote monitoring device as claimed in claim 1;
a weighing scale;
an oxygen meter; the weighing scale and the oxygen meter being connected to the remote monitoring device; and
a remote server connected to the remote monitoring device and configured to obtain, store and display the history of the measurements of the patient.

7. A method for remotely monitoring a patient suffering from heart failure, the method being intended to be implemented by a remote monitoring device and comprising the following steps:
obtaining, via a first communication interface with a weighing scale, measurements of the weight of the patient,
obtaining, via a second communication interface with an oxygen meter, measurements of the heart rate and of the blood oxygen saturation level of the patient;
triggering of an alert when the measurements obtained indicate a probable onset of cardiac decompensation in the patient;
obtaining predetermined values comprising:
a first weight limit increase;
a second weight limit increase strictly less than the first weight limit increase;
a first blood oxygen saturation level;
a second blood oxygen saturation level strictly greater than the first blood oxygen saturation level;
a first heart rate; and
a second heart rate strictly less than the first heart rate,
determining, from the obtained measurements, whether any one of the following first conditions is fulfilled:
the weight has increased by a value strictly greater than the first weight limit increase relative to the nominal weight of the patient;
the weight has increased, between two measurements taken over a predetermined period strictly greater than 2 days, by a value strictly greater than the first weight limit increase;
the blood oxygen saturation level is strictly less than the first blood oxygen saturation level;
the heart rate is strictly greater than the first heart rate;
triggering of an alert signaling that an onset of cardiac decompensation is probable when at least one of the first conditions is fulfilled;
determining, from the obtained measurements, whether any one of the following second conditions is fulfilled:
the weight has increased between two measurements taken over 2 consecutive days, by a value strictly greater than the second weight limit increase;
the blood oxygen saturation level is strictly less than the second blood oxygen saturation level;
the heart rate is strictly greater than the second heart rate;
triggering of an alert signaling that an onset of cardiac decompensation is probable when two of the second conditions are fulfilled simultaneously, while none of the first conditions is fulfilled.

8. The method as claimed in claim 7, wherein the nominal weight corresponds to a first weight measurement, a first measurement after a resetting or a first measurement after a period without measurement of more than 4 days.

9. The method as claimed in claim 7, wherein
the first weight limit increase is equal to 4 kg;
the second weight limit increase is equal to 2 kg;
the predetermined period is 4 days;
the first blood oxygen saturation level is equal to 90%;
the second blood oxygen saturation level is equal to 95%;
the first heart rate is equal to 110 beats/min;
the second heart rate is equal to 90 beats/min.

10. A non-transient computer readable medium containing program instructions for causing a computer to perform the method of claim 7.
